(19) United States
(12) Patent Application Publication
(10) Pub. No.: US 2014/0123388 A1
(43) Pub. Date: May 8, 2014

(54) AUTOMATED INITIAL SETUP POSITIONING FOR SPEEDING PATIENT THROUGHPUT

(71) Applicant: Reto W. Filiberti, Steinhausen (CH)

(72) Inventor: Reto W. Filiberti, Steinhausen (CH)

(21) Appl. No.: 13/669,030

(22) Filed: Nov. 5, 2012

(51) Int. Cl. A61B 6/04

(52) U.S. Cl. 5/601

(57) ABSTRACT

Methods, apparati, and computer-readable media for automatically moving a patient (1) to an initial setup position. A method embodiment of the present invention entails the steps of pointing to at least one spot (8) on the body of the patient (1) when the patient (1) is located at the initial setup position at a first time point; recording spatial coordinate information for the at least one spot (8); and automatically moving the patient (1) to the initial setup position at a subsequent time point.

### TECHNICAL FIELD

This invention pertains to the field of positioning a patient into an initial setup position for multiple fractions of radiation therapy or diagnosis.

### BACKGROUND ART

Modern diagnosis and radiation therapy machines have improved the quality of health care for miliions of patients. However, these machines are very expensive, and thus it is important to be able to move the patient into the identical initial setup position quickly when muitipte fractions are used. The present invention considerably speeds up this process, by (in one embodiment) using pointers, such as infrared pointers, corresponding detectors, and automated means to move the patient's couch into the initial setup position for each of a series of fractions.

### DISCLOSURE OF INVENTION

Methods, apparati, and computer-readable media for automatically moving a patient (1) to an initial setup position. A method embodiment of the present invention entails the steps of pointing to at least one spot (8) on the body of the patient (1) when the patient (1) is located at the initial setup position at a first time point; recording spatial coordinate information for the at least one spot (8); and automatically moving the patient (1) to the initial setup position at a subsequent time point.

### BRIEF DESCRIPTION OF THE DRAWINGS

These and other more detailed and specific objects and features of the present invention are more fully disclosed in the following specification, reference being had to the accompanying drawings, in which:

FIG. 1 is a side view showing a patient 1 in an initial setup position.

FIG. 2 is a side view showing the patient 1 in a starting position other than the initial setup position. In this case, the starting position is a patient loading position.

FIG. 3 is a side view of apparatus used in the present invention, using a different gantry 31 than is shown in FIG. 2.

FIG. 4 is a top view of the apparatus depicted in FIG. 3.

FIG. 5 is a top view of a patient 1 upon whom three spots 8 have been marked.

FIG. 6 is a top view of an embodiment of the present invention in which the invention is used in conjunction with a gating system using optical scanning detectors 61.

### DETAILED DESCRIPTION OF PREFERRED EMBODIMENTS

As used herein, the following terms have the following meanings:

“Patient” means a human or other animal.

“Fraction” is a time period during which a dose of radiation therapy is administered to a patient 1, or, in the case where this term pertains to a diagnostic procedure rather than a therapeutic procedure, one of the times during which the diagnostic procedure is performed.

“Isocenter” is the intersection of the beam axis 5 and the gantry rotation axis 6.

“Initial setup position” is the location where the patient 1 is correctly positioned for the therapy or diagnosis. In the case where a tumor 13 is being treated, the tumor 13 is located at the isocenter 4 when the patient is at the initial setup position.

“Starting position” is a position where the patient 1 is initially located on couch 2 prior to therapy or diagnosis, prior to being moved to the initial setup position.

This invention can be used for diagnosis where there are multiple fractions, as well as for therapy. For example, the diagnosis can use ultrasound, magnetic resonance imaging, positron emission tomography, computed tomography, or any other imaging technique to diagnose a medical problem within a patient 1. However, for purposes of illustration only, the bulk of this written description discusses the case where the invention is used for radiation therapy, including image guided radiation therapy (IGRT). It will be understood by those of ordinary skill in the art that the detailed descriptions herein can be equally applied to fractionated diagnosis, mutatis mutandis.

The radiation therapy can be any type, including without limitation photon therapy, electron therapy, proton therapy, and heavy ion therapy.

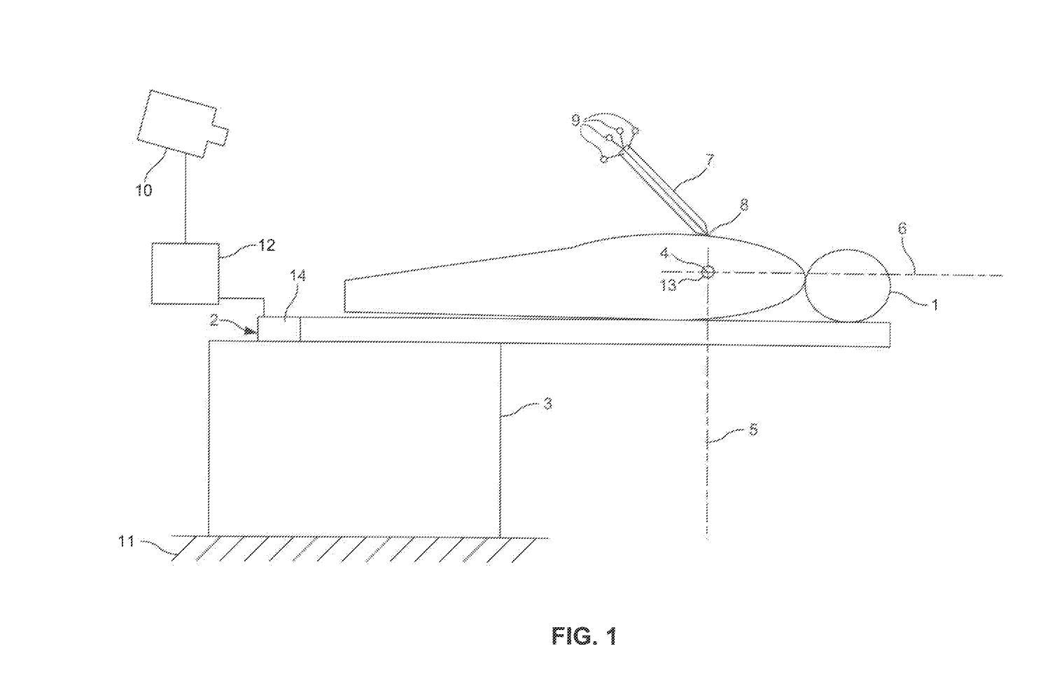

FIG. 1 illustrates a moment in a method of the present invention in which the patient 1 is at an initial setup position for a first treatment fraction. The patient 1 has an internal tumor 13, which is positioned at the isocenter 4. The patient is lying on a treatment couch 2 that is supported by a stand 3 that is physically mounted to the floor 11 of the treatment room. Couch 2 can be any type of couch. For example, couch 2 can be a robotic couch, i.e., one that is operated by remote control. Robotic couch 2 can have linear motions, angular motions, or a combination of linear and angular motions. Alternatively, couch 2 can be a non-robotic couch having a float mode in which the couch 2 can be made to move laterally and longitudinally, manually by a human operator. Couch 2 can be a robotic couch having motorized floating motions, wherein force sensitive handles control motorized movements to mimic float mode as a way to have floating motions on a robotic couch. The couch 2 illustrated herein is equipped with one or more motors 14 that accept commands from command module 12 in order to position couch 2 with up to six degrees of freedom (vertical, lateral, and longitudinal directions; and rotational, pitch, and roll axes) about isocenter 4. The commands cause the couch 2 to move automatically to desired positions.

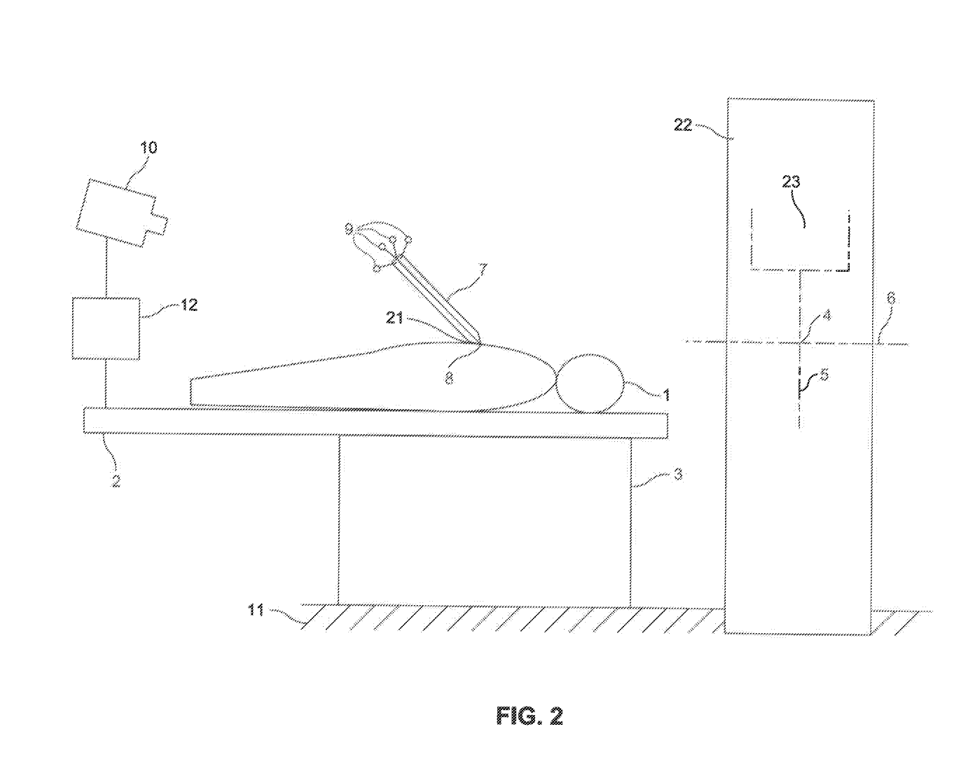

One or more spots (marks) 8 are made on the surface of the patient's body 1 to assist in the automatic positioning. These one or more spots 8 are located in a known geometrical relationship with respect to the location of the tumor 13. The spots 8 are normally visible to the human eye, and may be, for example, marks made with a pen or Sharpie™. In one embodiment of the present invention, a pointer 7 is placed against the body 1 so that the tip 21 of the pointer (see FIG. 2) points to the at least one spot 8. Pointer 7 can be an illuminator that illuminates light on the spot(s) 8. Alternatively, pointer 7 can be just a physical pointer. In still other embodiments, a combination of optical and non-optical pointers 7 are used.

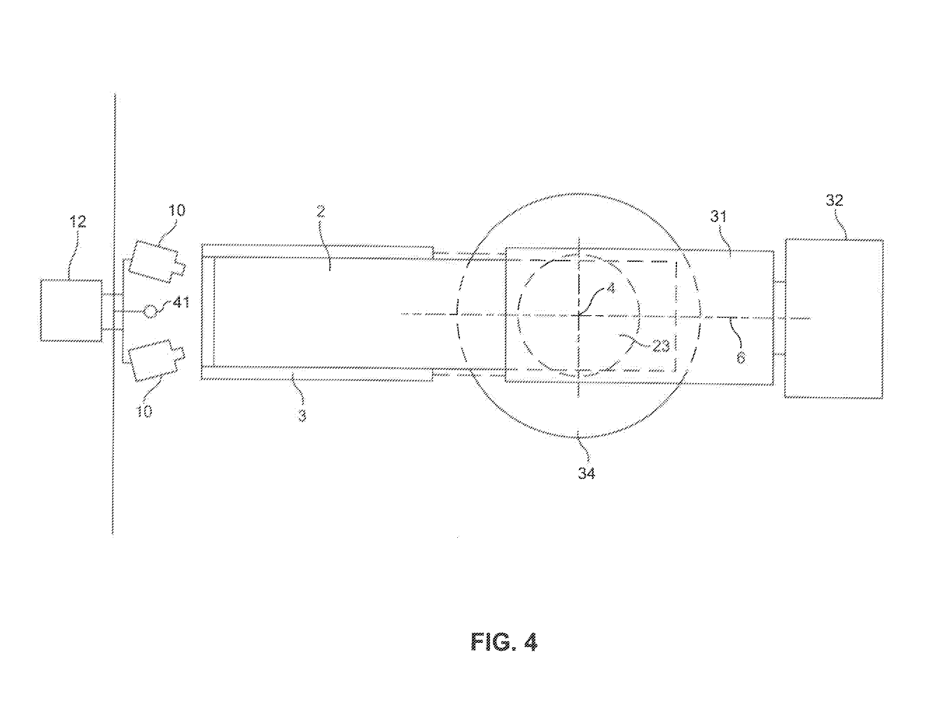

Tip 21 can be placed at spot(s) 8 manually by a human operator, or in another embodiment, pointer 7 can be coupled to a robotic arm (not iiiustrated) that is commanded by remote control to place pointer 7 in the proper location(s). The end of pointer 7 distal from tip 21 is affixed with one or several markers 9. In an embodiment in which pointer 7 is an infrared pointer, markers 9 can be sources of infrared luminosity which is detectable by one or more detection devices (cameras) 10 that are normally positioned at upper regions of the treatment room. Alternatively, as illustrated in FIG. 4, markers 9 are not active markers that themselves emanate optical luminosity, but rather passive markers that reflect optical luminosity that emanates from an iiluminator 41 that is present in the treatment room.

The reason for having more than one marker 9 is to provide for recordation of the angular orientation of pointer 7, which could not be achieved with the use of just one marker 9. The geometry of pointer 7 and markers 9 are known to the optical system command module 12. Therefore, command module 12 can calculate the location of pointer tip 21, based on the position and angular orientation of the markers 9.

Using more than one detection device 10, such as the two detection devices 10 that are illustrated in FIG. 4, makes for better angular accuracy in the detection of the luminosity, which gives a more accurate absolute position of the markers 9 and therefore also the position of tip 21.

In embodiments where pointer 7 is an illuminator, the light that is used for pointer 7 and detectors 10 can be light in any wavelength band anywhere from infrared to ultraviolet, inclusively. Visible light can be used, but is typically not used, because it might interfere with the ambient light in the treatment room. Other signaling can be used, such as a laser pointer 7 and a laser camera 10, which offers better resolution than with infrared pointers 7, but at higher cost.

In still other embodiments, ultrasound or RF (radio frequency radiation) can be used in lieu of optical pointing. For ultrasound, transmitters or transponders are located on or proximate to the patient 1, and corresponding ultrasound detection devices 10 are used to receive the ultrasound radiation. For RF, transmitters or active or passive transponders are located on or proximate to the patient 1, and corresponding RF detection devices 10 are used to receive the RF radiation. These transmitters and transponders can remain on the patient 1 over the course of multiple fractions. These transmitters and transponders do not have to be located along the beam axis 5, as long as there is a known geometrical relationship between the transmitters and transponders, and the location of the tumor 13. Thus, the transmitters and transponders do not have to be affixed to the patient 1, but rather can be merely proximate to the patient 1.

In an alternative embodiment, a pointer 7 is not used, but rather an optical marker (which can be infrared or ultraviolet, active or passive) is placed temporarily on the patient's body 1 for the setup, the whole fraction, multiple fractions, or the entire course of treatment.

In yet another alternative embodiment, the location of the initial setup position is determined not by a pointer but rather in a three-dimensional planning process that is performed prior to the first treatment fraction. In this embodiment, command module 12 can readily obtain enough information to enable module 12 to position couch 2 (and therefore patient 1) with all six of the aforesaid degrees of freedom.

This invention can be used in conjunction with image guided radiation therapy (IGRT), in which X-rays or other images are taken when the patient 1 is in the initial setup position, or close thereto. These images are then matched with reference images for fine-tuning the initial setup position. This can be done remotely and/or automatically.

Whatever type of signaling is used, the position information obtained by the at least one detection device 10 is fed to command module 12, which is coupled to the detector(s) 10 and also has access to a prestored representation of the three-dimensional coordinates of the treatment room. Then, command module 12, taking into account the known geometrical relationship between the at least one spot 8 and the location of the tumor 13, calculates, typically in a fixed three-dimensional coordinate system that is correlated with the three-dimensional coordinates of the treatment room, a three-dimensional vector representing where couch 2 needs to be moved in order to place the patient 1 into the initial setup position for fractions other than the first fraction. Using these calculations, command module 12 sends appropriate commands to at least one motor 14 associated with couch 2 to cause couch 2 to automatically place the patient in the initial setup position for said subsequent fraction.

Command module 12 can comprise any combination of hardware, firmware, and/or software. Any such software modules located within command module 12 can be resident on one or more computer-readable media, such as at least one hard disk, floppy disk, optical disk, DVD, etc., containing computer program instructions needed or useful to carry out the above-described calculations and to perform the above-described commands.

FIG. 2 illustrates an embodiment in which a ring gantry 22 is used to provide the therapeutic radiation. Gantry 22 is able to rotate around axis 6, and is mounted via bearings to a fixedly mounted stand (not shown) on floor 11. A collimator 23 within gantry 22 shapes the radiation emanating from within gantry 22 along beam axis 5 in the direction of the tumor 13. The degree of collimation can vary from fraction to fraction, and can also vary within a fraction.

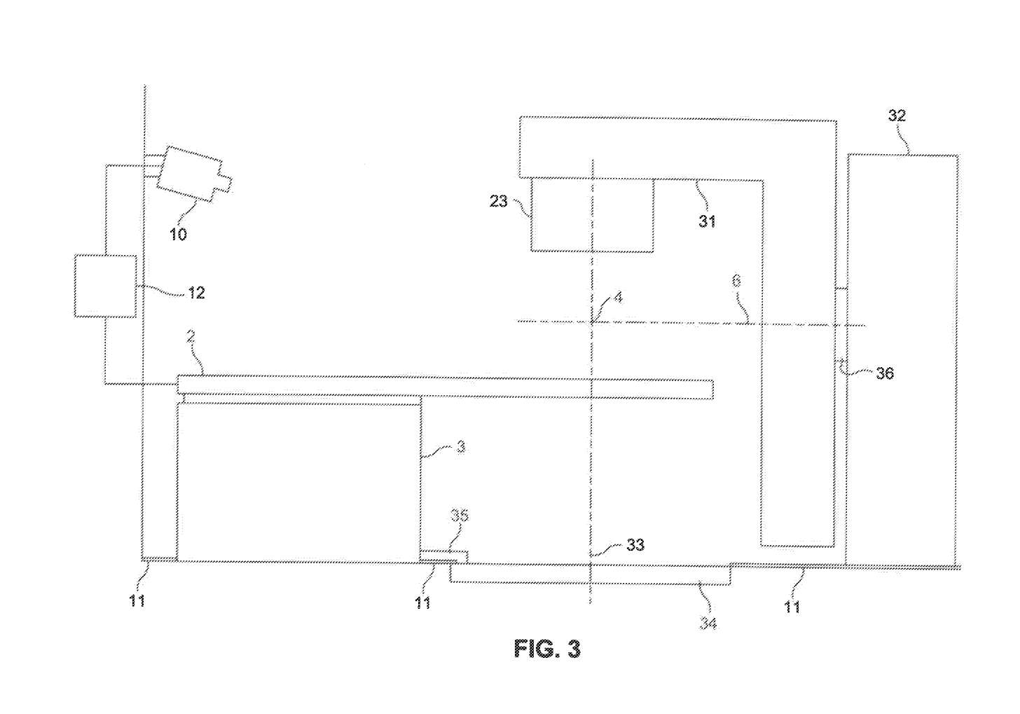

FIG. 3 illustrates an alternative embodiment in which gantry 31 is an L-shaped gantry rather than a ring gantry 22. L-shaped gantry 31 is rotationally mounted via rotator 38 to a massive stand 32 that is fixedly mounted to floor 11. Gantry 31 rotates about gantry axis 6. In this embodiment, collimator 23 protrudes from the horizontal arm of gantry 31. FIG. 3 also illustrates that couch 2 is rotatable about a couch rotation axis 33. This can be accomplished by couch rotation turntable 34, which is mounted beneath floor 11 and coupled to couch stand 3 via a rigid connector arm 35. In that case, couch stand 3, which imparts vertical movement to couch 2, is not fixedly connected to floor 11.

FIG. 4 illustrates a top view of the arrangement depicted in FIG. 3.

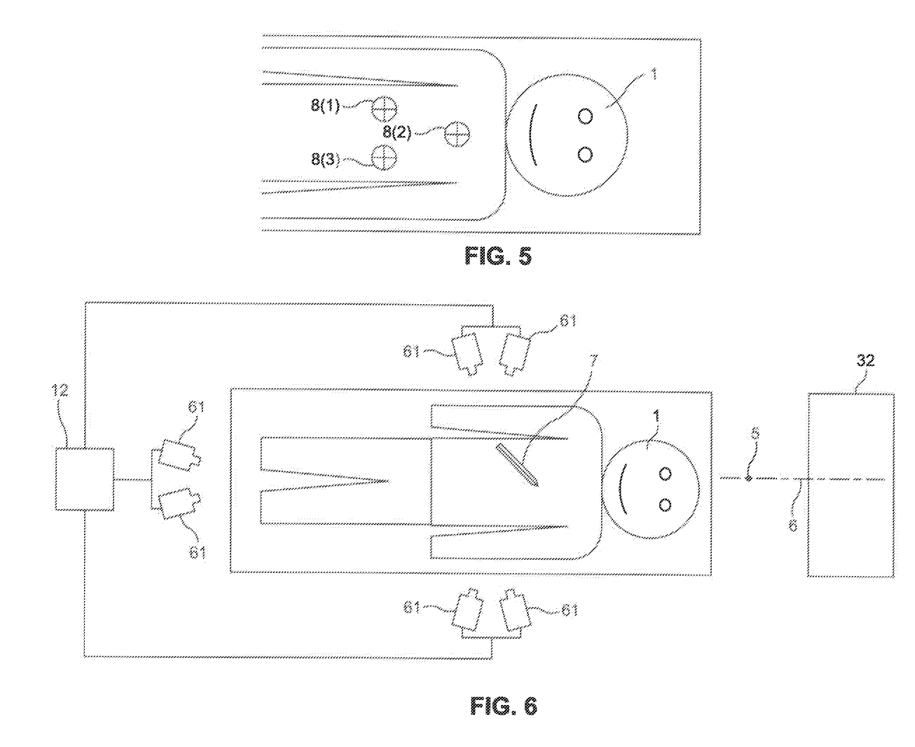

FIG. 5 illustrates an embodiment of the invention in which multiple spots 8, in this case three of them, are marked on the patient 1. This technique provides greater angular accuracy compared with the embodiment in which just one spot 8 is used. It also allows command module 12 to obtain information to position couch 2 (and therefore patient 1) with all six of the aforesaid degrees of freedom. In one embodiment, a confirmation button (not illustrated) can be integrated into pointer 7. Alternatively, the confirmation button can be located on couch 2 or anyplace else. Signals from the confirmation button are read by each detection device 10 or by a separate device that is also coupled to command module 12. The operator points to point 8(1) and then presses the confirmation button. This tells command module 12 that information from spot 8(1) needs to be recorded for module 12's subsequent calculations. The operator then points in turn to spots 8(2) and 8(3), each time pressing the confirmation button, again signaling command module 12 that the information from spots 8(2) and 8(3) needs to be recorded.

FIG. 6 illustrates the use of the present invention in conjunction with a gating system employing a surfacing scanning device. Such a gating system for radiation therapy comprises the use of an optical or video imaging system to measure regular physiological movement of a patient's body. The image data are used to quantify voluntary or involuntary motions of the patient that may affect the delivery of radiation to the target tissue. A gating signal is generated to suspend delivery of radiation upon certain threshold events detected in the physiological movement. Such gating systems are more fully described in U.S. Pat. Nos. 6,690,965 and 6,279,579, the disclosures of which are hereby incorporated by reference in their entireties into the present patent application.

With respect to the present invention, the gating system uses one or several optical scanning detectors 61 that detect the above-described motion on the surface of the patient's body 1. Detectors 61 constantly scan a projected grid (or other pattern) on said surface 1 in three dimensions, calculate three-dimensional differences of patient position with respect to time, and give a pseudo three-dimensional representation to a human operator on a two-dimensional display. The gating system is calibrated to room coordinates, as are embodiments of the present invention that are described above. The results of the scan of the patient's surface 1 performed by detectors 61 can be displayed in near realtime on one or more displays that are coupled to the detectors 61. These displays can be located inside and/or outside the treatment room.

The gating system uses mathematical correlation models to correlate between the motion of the patient's surface 1 and the motion of the tumor 13. Such correlation models are defined and/or verified during treatment planning and/or prior to each fraction, and are used to inform the operator of the treatment control system when the patient's breathing has moved tumor 13 to the point where tumor 13 is too far out of position for safe and/or effective therapy. Typically, the information given to the operator has a resolution of about 2 mm and covers the whole body 1. When the tumor 13 is too far out of position, the gating system either sends an alarm to the operator, or automatically activates a switch to shut off or pause the flow of radiation emanating from the gantry 22, 31.

One way in which the present invention can be used with the gating system is for command module 12 to convey to the gating system where the spots 8 and/or the markers of the gating system are, thereby telling the gating system what locations it should be monitoring, and how this monitoring is proceeding. Additionally, command module 12 performs the duties described above regarding determining where the patient 1 needs to be moved in order for the patient 1 to be in the initial setup position, and issuing appropriate commands to motors(s) 14.

The above description is included to illustrate the operation of preferred embodiments, and is not meant to limit the scope of the invention. The scope of the invention is to be limited only by the following claims. From the above description, many variations will be apparent to one skilled in the art that would yet be encompassed by the spirit and scope of the present invention.

What is claimed is:

1. A method for automatically moving a patient to an initial setup position, said method comprising the steps of:
identifying a plurality of spots on the patient's body at a first time point when the patient is positioned at the initial setup position;
recording spatial coordinate information of the spots as the patient is positioned at said initial setup position;
taking into account the location of the spots from the first time point, automatically moving the patient to the initial setup position at a second time point; and
as the spots are pointed to in succession, sending a signal to the recording means at each pointed-to spot.

2. The method of claim 1 wherein:
the identifying step is performed by a pointing device having at least one optical marker; and
the recording step uses at least one optical detector.

3. The method of claim 2 wherein each optical marker and optical detector is operable in a wavelength band between infrared and ultraviolet, inclusively.

4. The method of claim 2 wherein the markers are active markers.

5. The method of claim 2 wherein the markers are passive markers.

6. The method of claim 1 wherein the identifying and pointing is performed by a laser pointer; and
the recording step uses at least one laser camera.

7. The method of claim 1 wherein the recording step uses at least one RF detector that measures RF emanating from at least one RF transmitter, or active or passive RF transponder, located on or proximate to the patient.

8. The method of claim 1 wherein the recording step uses at least one ultrasound detector that measures an ultrasound signal emanating from at least one ultrasound transmitter or transponder located on or proximate to the patient.

9. The method of claim 1 wherein each spot is visible to a human.

10. The method of claim 1 wherein the moving step is directed by at least one software module coupled to at least one motor in a couch to which the patient is coupled.

11. The method of claim 1 wherein there are a plurality of spots marked on the patient's body; and
the recording step records spatial coordinate information for each spot, whereby angular corrections are made.

12. The method of claim 11 wherein the recording step obtains information sufficient to enable the moving step to move the patient with six degrees of freedom.

13. (canceled)

14. The method of claim 1 wherein the recording step records three-dimensional coordinates of the spots with respect to a fixed-coordinate system of a room in which the patient is located.

15. The method of claim 1 wherein the time points correspond to fractions of radiation therapy.

16. The method of claim 15 wherein the radiation therapy is image guided radiation therapy.

17. The method of claim 1 wherein the initial setup position is for patient diagnosis.

18. The method of claim 17 wherein the diagnosis is from the group of diagnoses consisting of ultrasound, magnetic resonance imaging, positron emission tomography, and computed tomography.

19. The method of claim 1 wherein at least one optical scanning detector continuously scans a surface of the patient in three dimensions; and
the recording step records three-dimensional coordinates of optical scanning markers using the same coordinate system as for the recording of the position of the spots; wherein
the at least one optical scanning detector tracks the markers while continuously scanning the surface.

20. The method of claim 19 wherein the at least one optical scanning ietector is coupled to a display adapted to display the surface scan in near realtime.

21. Apparatus for moving a patient to an initial setup position, said apparatus comprising:
identification means for identifying at least one spot on the patient's body at a first time point when the patient is in the initial setup position;
at least one recording device adapted to receive signals from the identification means and to record spatial coordinates of the at least one spot as the patient is in the initial setup position; and
coupled to the at least one recording device, moving means for automatically moving the patient to the initial setup position at a subsequent time point; wherein
the moving means is configured to move the patient with up to six degrees of freedom, comprising vertical, lateral, and longitudinal directions, and rotational, pitch, and roll axes about an isocenter.

22. The apparatus of claim 21 wherein the moving means comprises means for calculating a three-dimensional vector connecting the patient's current position with the initial setup position.

23. The apparatus of claim 21 wherein:
the identification means comprises a pointer having infrared markers; and
each recording device comprises an infrared detector.

24. The apparatus of claim 21 wherein the moving means comprises:
at least one software module coupled to the at least one recording device; and
at least one motor adapted to move a couch upon which the patient lies, said at least one motor adapted to respond to commands from the at least one software module.

25.-27. (canceled)
